(19) United States
(12) Patent Application Publication
(10) Pub. No.: US 2011/0046353 A1
(43) Pub. Date: Feb. 24, 2011

(54) Purification Process for Anitbody Fragments Using Derivatized Triazines as Affinity Ligands

(76) Inventor: John Macdonald Liddell, Cleveland (GB)

Correspondence Address: MORGAN LEWIS & BOCKIUS LLP, 1111 PENNSYLVANIA AVENUE NW, WASHINGTON, DC, 20004, US

(21) Appl. No.: 12/990,622

(22) PCT Filed: May 7, 2009
(86) PCT No.: PCT/GB2009/001126

§ 371 Date: Nov. 1, 2010

(30) Foreign Application Priority Data
May 16, 2008 (GB) 0808908.8

(51) Int. Cl. C07K 16/00

(52) U.S. Cl. 530/387.1

(57) ABSTRACT

A process for the separation of a fragment antibody from a medium is provided. The process comprises contacting the medium comprising the fragment antibody with a synthetic affinity ligand attached to a support matrix under conditions whereby the fragment antibody binds to the synthetic affinity ligand. The synthetic affinity ligand has the formula (I): wherein Q represent an attachment to a solid support matrix, optionally via a spacer group; A and B are each independently —Y-phenyl or —Y-naphthyl groups substituted with one or more substituents capable of hydrogen bonding, preferably one or more of —OH, —SH or —CO₂H groups; each Y independently represents —NR—, —O— or —S—; and each R independently represents H or a C₁₋₄ alkyl group.

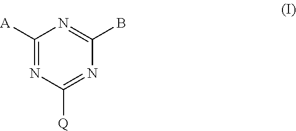

The present invention concerns a process for the purification of fragment antibodies (fAbs).

Fragment antibodies (fAbs) are of increasing importance in a range of therapeutic areas. One of the most important methods of producing fAbs is by recombinant technology. Such techniques use a host cell to express the desired fAb, which is then separated from the production medium and purified. Purification is commonly achieved by chromatography, and is typically a complicated, multi-step process. This complexity inevitably serves to limit the yields of fAb that can be obtained, and significantly slows the manufacturing process. Accordingly, it would be desirable to identify purification methods amenable to simpler operation.

Many whole antibodies are purified by using Protein A affinity chromatography. However, Protein A is recognised as having a number of deficiencies, including poor stability under sometimes harsh process conditions, denaturation, high cost and regulatory concerns arising from the fact that Protein A is itself biologically-sourced material. Synthetic affinity ligands have therefore been developed as alternative for the purification of antibodies having an affinity for Protein A.

fAbs are not purified by Protein A affinity chromatography, because the fAbs do not have an affinity for Protein A, and therefore do not bind.

According to one aspect of the present invention, there is provided a process for the separation of a fAb from a medium which comprises contacting the medium comprising the fAb with a synthetic affinity ligand attached to a support matrix under conditions whereby the fAb binds to the synthetic affinity ligand, wherein the synthetic affinity ligand has the formula:

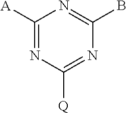

wherein
Q represents an attachment to a solid support matrix, optionally via a spacer group;
A and B are each independently —Y-phenyl or —Y-naphthyl groups substituted with one or more substituents capable of hydrogen bonding, preferably one or more of —OH, —SH or —CO₂H groups;
each Y independently represents —NR—, —O— or —S—; and
each R independently represents H or a C₁₋₄ alkyl group.

fAbs which can be purified by the process of the present invention are sections of antibodies comprising an immunoglobulin domain or an assembly of immunoglobulin domains and which are capable of binding to an antigen, and which, in many embodiments, comprise at least one heavy chain, commonly a V_{H} chain, or a functional fragment thereof, or a light chain, commonly a V_{L} chain, or a functional fragment thereof, together with at least one other chain. In certain embodiments, the fAb comprises a heavy chain and a light chain, each chain being made up of a constant domain and a variable domain, such as a Fab. In other embodiments, the fAb comprises two or more domains, typically a combination of either the variable and constant domains of either heavy or light chains, combinations of variable domain from two heavy chains, combinations of variable domains from two light chains, or a combination of the variable domain from a light chain and the variable domain from a heavy chain. In some embodiments, the fAb comprises the V_{H} and V_{L} domains joined by flexible polypeptide linker preventing dissociation (single chain Fv, scFv). In yet further embodiments, the fAb comprises a single domain, or a fragment thereof, typically either the variable heavy chain or a fragment thereof, or the variable light chain or a fragment thereof. In still further embodiments, the fAb is a multimeric format, such as a bis scFv, Fab₂, Fab₃, minibody, diabody, triabody, tetrabody or tandab.

Examples of fAbs that can be purified by the process of the present invention include protein or polypeptide constructs comprising a combined heavy chain and a light chain, each chain being made up of a constant domain and a variable domain where such immunoglobulin light and heavy chains interact to form a single functional antigen-binding site.

Further examples include V_{H} chain-based domain antibodies, being polypeptides which are capable of binding to a target, the polypeptide comprising at least one binding domain, wherein the binding domain is a single variable domain of a variable heavy chain antibody or a functional fragment thereof.

Yet further examples include V_{L} chain-based domain antibodies, being polypeptides which are capable of binding to a target, the polypeptide comprising at least one binding domain, wherein the binding domain is a single variable domain of a variable light chain antibody or a functional fragment thereof.

Most preferably, the fAbs are produced recombinantly, for example by expression in a host cell, for example in a prokaryotic host such as E. coli or in a eukaryotic host such as Pichia pastoris.

Preferred affinity ligands are compounds of formula:

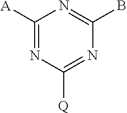

wherein Q represents an attachment to a solid support matrix, optionally via a spacer group, and A and B are each independently —NH-phenyl or —NH-naphthyl groups substituted with one or more of —OH, —SH or —CO₂H groups. When either of A or B represent phenyl, a substituent, most preferably —OH, is preferably located at the position meta or para to the bond to the —NH moiety. Especially preferred affinity ligands include compounds of formula:

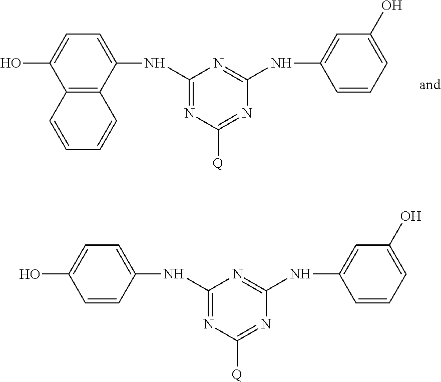

wherein Q represents an attachment to a solid support matrix, optionally via a spacer group.

Spacer groups which can be represented by Q include optionally substituted aminoalkylamino moieties, such as a group of formula —NH—(CH₂)ₙNH-G where n is a positive integer up to 12, preferably from 2-6 and G is a solid support matrix; a group of formula —NH—(CH₂)ₙO-G where n and G are as previously defined; a group of formula —O—(CH₂)ₙO-G where n and G are as previously defined; a group of formula —O—(CH₂CH₂)ₙO-G where n and G are as previously defined; a group of formula —NH—(CH₂)ₙO-G where n and G are as previously defined; a group of formula —NH—(CH₂)ₙNH—(CH₂)ₓO-G where n and G are as previously defined, and x is from 1 to 6. One or more of the —CH₂— moieties my be substituted by one or more substituents, for example OH or NH₂ groups.

Solid support matrices to which the affinity ligands can be attached are well known in the field of affinity chromatography, and include synthetic polymers, such as polyacrylamide, polyvinylalcohol or polystyrene, especially cross linked synthetic polymers; inorganic supports, such as silica-based supports; and particularly polysaccharide supports, for example starch, cellulose or agarose.

In certain embodiments, excellent results have been achieved using the supported affinity ligands commercially available from Prometic Biosciences under the tradenames MAbsorbent A1P and MAbsorbent A2P.

Contact between the medium containing a fAb and the supported affinity ligands is effected under conditions where the fAb binds to the affinity ligand. In many embodiments, an aqueous solution comprising fAb at about neutral pH, for example a pH from about 6 to 8, for example 6.5 to 7.5, and especially a pH of 7. In some embodiments, the aqueous solution has a high ionic strength, such as from 75 to 125 mS/cm, but in many embodiments, the aqueous solution preferably has a low ionic strength, such as an ionic strength of less than 50 mS/cm, for example between 10 and 40 mS/cm, and preferably about 30 mS/cm, such as from 27 to 33 mS/cm. Contact is preferably continued until substantially all of the fAb is bound to the affinity ligand. Many impurities which may be present in the medium comprising the fAb do not bind to the affinity ligand and therefore remain in the medium.

Commonly, the supported affinity ligand is employed in a chromatography column, and the medium comprising the fAb is flowed through the column. A single pass through the column may be employed, or as alternatives, the medium can be recirculated through the column. Two or more columns may be employed in sequence.

The support comprising the bound fAb may be washed with one or more wash solutions under conditions where the fAb remains bound, for example, depending upon the nature of the fAb, employing aqueous buffers of low ionic strength, and about neutral pH, or employing buffers of about neutral pH and an ionic strength corresponding to, or higher than, that of the aqueous solution employed to load the fAb.

The fAb can then be separated from the affinity ligand by contact with a solution which causes the fAb to be released from the ligand, for example by varying the ionic strength. In many embodiments, the elution solvent comprises an aqueous solution having a lower pH than the medium from which the fAb was attached to the ligand, for example an buffer solution having a pH in the range of from 2 to 4. If desired, an elution gradient can be employed.

According to a second aspect of the present invention, there is provided a process for the preparation of a fAb comprising:
a) preparing a fAb by recombinant technology to produce a medium comprising fAb;
b) separation of the fAb from the medium by a process comprising contacting the medium comprising the fAb with a synthetic affinity ligand attached to a support matrix under conditions whereby the fAb binds to the synthetic affinity ligand, wherein the synthetic affinity ligand has the formula:

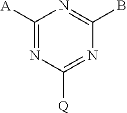

wherein
Q represents an attachment to a solid support matrix, optionally via a spacer group;
A and B are each independently Y-phenyl or Y-naphthyl groups substituted with one or more substituents capable of hydrogen bonding, preferably one or more of —OH, —SH or —CO₂H groups;
each Y independently represents —NR—, —O— or —S—; and
each R independently represents H or a C₁₋₄ alkyl group ; and
c) releasing the fAb from the affinity ligand.

fAbs produced by the process according to the second aspect of the present invention may be subjected to further purification steps if desired, for example one or more of ion exchange chromatography; chromatography based on hydrophobicity, such as HIC, reverse phase chromatography, hydrophobic charge induction chromatography, or mixed mode chromatography; or size-based purifications such as gel filtration.

The present invention is illustrated without limitation by the following examples.

### EXAMPLE 1

An fAb (from the monoclonal anti lysozyme antibody D1.3) was produced by periplasmic expression in a recombinant E coli strain. The fAb with a total molecular weight 47.4 kDa (two chains—a heavy chain comprising a variable light domain with contestant domain attached and a heavy chain comprising a variable light domain with constant domain attached) was secreted into the cell periplasm and subsequently into the fermentation growth medium. At the end of fermentation levels of D1.3 present in the fermenter supernatant were around 100 mg/L.

Initial isolation of fAbD1.3 involved centrifugation to remove cellular material with subsequent filtration through a 0.45/0.2 micron filter. The resulting clarified solution had a conductivity of 29.3 mS/cm and a pH of 6.7.

Two 1.6 cm diameter columns were packed, one with Prometic Biosciences A1P and the second with Prometic Biosciences A2P biomimetic protein A chromatography media to a bed height of 2.5 cm in each case. The column packing was assessed by asymmetry and HETP.

For each column a similar protocol was followed:

| | |
|---|---|
| Equilibration wash | 50 mM sodium phosphate pH 7 |
| D1.3 binding | |
| Post load wash | 50 mM sodium phosphate pH 7 |
| Elution | 50 mM sodium citrate pH 3 |
| Regeneration | 0.5M NaOH |

In each case no conditioning of the D1.3 load material took place other than to adjust the pH to 7. A single elution peak was obtained with the 50 mM citrate elution buffer in each case.

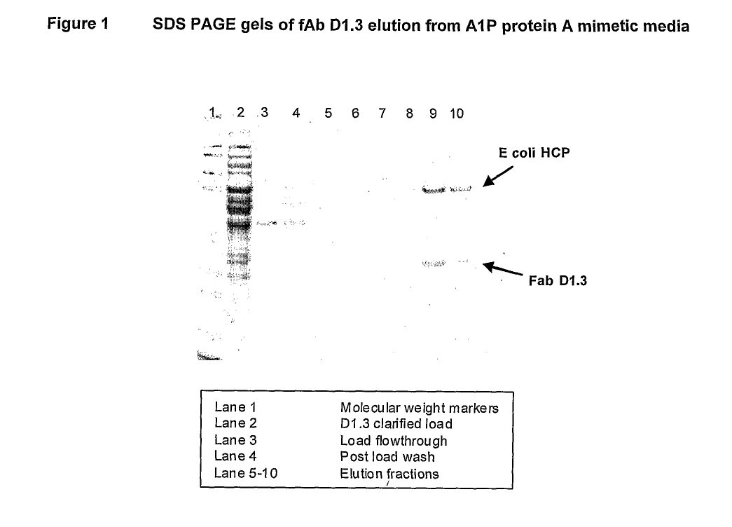

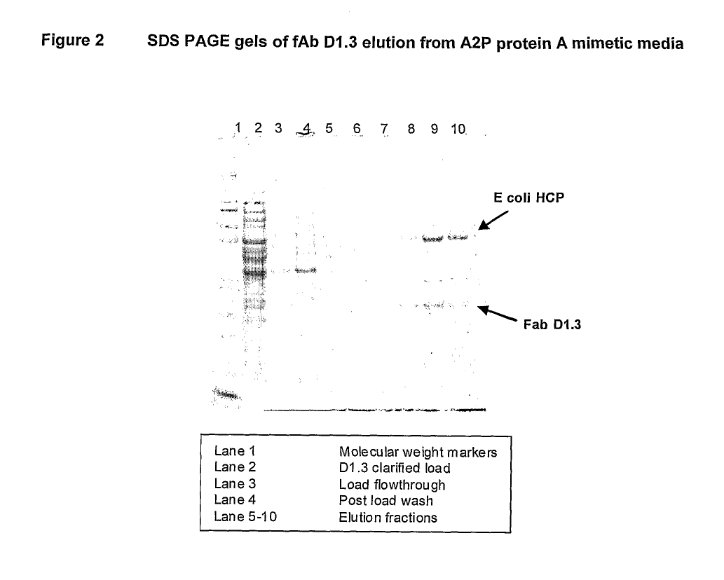

SDS PAGE gels of the elution fractions indicted that a high degree of purification of the fAbD1.3 was obtained in each case (see FIG. 1 and FIG. 2).

### COMPARATIVE EXAMPLE 2

A comparative experiment was carried out attempting to bind fAbD1.3 to a protein A media. A sample of the same fAbD1.3 as used in Example 1 was applied to a 1 ml MabSelect (GE Healthcare) Protein A column. fAbD1.3 fermenter supernate was centrifuged to remove cells and filtered through a 0.45/0.2 micron filter and adjusted to pH 7. The conductivity of the load material was 29 mS/cm.

The chromatography protocol followed was that recommended by the manufacturer for IgG binding:

| | |
|---|---|
| Equilibration wash | 10 mM sodium phosphate, 150 mM NaCl pH 7 |
| D1.3 loading | |
| Post load wash | 10 mM sodium phosphate, 150 mM NaCl pH 7 |
| Elution | 100 mM sodium citrate pH 3 |
| Regeneration | 10 mM NaOH |

No elution peak was seen when the 100 mM citrate elution buffer was applied. Examination of elution fractions by SDS PAGE failed to detect fAbD1.3 in the elution wash fractions confirming that fabD1.3 did not bind to protein A media.

### EXAMPLE 3

A V_{L} based domain fragment (an anti TNF domain, TAR1-5-19—sequence ID no:16 in FIG. 12 of International patent application WO 2005035572A2) was produced by periplasmic expression in a recombinant E coli strain. The domain with a total molecular weight 11.9 kDa was secreted into the cell periplasm and subsequently into the fermentation growth medium. At the end of fermentation levels of the anti TNF domain present in the fermenter supernatant was around 2.4 g/L.

Initial isolation of domain TAR1-5-19 involved centrifugation to remove cellular material with subsequent filtration through a 0.45/0.2 micron filter. The resulting clarified solution had a conductivity of ca 32 mS/cm and a pH of 7.2.

Two 1.6 cm diameter columns were packed, one with Prometic Biosciences A1P and the second with Prometic Biosciences A2P biomimetic protein A chromatography media to a bed height of 4.3 cm (A1P) and 2.3 cm (A2P). The column packing was assessed by asymmetry and HETP.

For each column a similar protocol was followed:

| | |
|---|---|
| Equilibration wash | 25 mM sodium phosphate pH 7 |
| TAR1-5-19 domain binding | |
| Post load wash | 25 mM sodium phosphate pH 7 |
| Elution | Linear gradient over 15CV from 25 mM sodium phosphate pH 7 to |
| | 25 mM sodium citrate pH 3 |
| Regeneration | 0.5M NaOH |

In each case no conditioning of the anti TNF domain load material took place other than to adjust the pH to 7. Binding of the anti TNF domain was assessed by SDS PAGE gels and was assessed to be at a level of 10-20 mg/ml.

### EXAMPLE 4

A V_{H} based domain fragment (anti hen egg white lysozyme domain HEL4—Jespers et al J Mol Biol (2004) 337 893-903) was produced by periplasmic expression in a recombinant E coli strain. The domain with a molecular weight 12.8 kDa was secreted into the cell periplasm and subsequently into the fermentation growth medium. At the end of fermentation levels of the HEL4 domain present in the fermenter supernatant was around 1.5 g/L.

Initial isolation of HEL4 involved centrifugation to remove cellular material with subsequent filtration through a 0.45/0.2 micron filter. The resulting clarified solution had a conductivity of ca 31 mS/cm and a pH of 6.9.

Two 1.6 cm diameter columns were packed, one with Prometic Biosciences A1P and the second with Prometic Biosciences A2P biomimetic protein A chromatography media to a bed height of 4.3 cm (A1P) and 2.3 cm (A2P). The column packing was assessed by asymmetry and HETP.

For each column a similar protocol was followed:

| | |
|---|---|
| Equilibration wash | 25 mM sodium phosphate pH 7 |
| HEL4 domain binding | |
| Post load wash | 25 mM sodium phosphate pH 7 |
| Elution | Linear gradient over 15CV from 25 mM sodium |
| | phosphate pH 7 to 25 mM sodium citrate pH 3 |
| Regeneration | 0.5M NaOH |

In each case no conditioning of the HEL4 domain load material took place other than to adjust the pH to 7. Binding of the HEL4 domain was demonstrated by SDS PAGE gels.

### EXAMPLE 5

A multivalent antibody fragment derived tandem antibody fragment (tandab composed of two chains each containing four domains (two V_{H} and two V_{L} domains in the format (V_{H}V_{L}V_{H}V_{L})₂ as described in Example 15 of International patent application WO2007/088371) was produced by periplasmic expression in a recombinant E coli strain. The tandab with an overall molecular weight of ca 100 kDa was secreted into the cell periplasm and subsequently into the fermentation growth medium. At the end of fermentation levels of the tandab present in the fermenter supernatant was estimated to be ca. 100 mg/L.

Initial isolation of the tandab involved centrifugation to remove cellular material with subsequent filtration through a 0.45/0.2 micron filter. The resulting clarified solution had a conductivity of ca 31 mS/cm and a pH of 6.9.

Two 0.7 cm diameter columns were packed, one with Prometic Biosciences A1P and the second with Prometic Biosciences A2P biomimetic protein A chromatography media to a bed height of 3 cm.

For each column a similar protocol was followed:

| | |
|---|---|
| Equilibration wash | 25 mM sodium phosphate pH 7 |
| Tandab antibody fragment binding | |
| Post load wash | 25 mM sodium phosphate pH 7 |
| Elution | 25 mM sodium citrate pH 3 |
| Regeneration | 0.5M NaOH |

In each case no conditioning of the tandab antibody fragment load material took place other than to adjust the pH to 7. Binding of the tandab antibody fragment was assessed by SDS PAGE gels and specific binding and elution was detected.

1-13. (canceled)

14. A process for the separation of a fragment antibody from a medium which comprises contacting the medium comprising the fragment antibody with a synthetic affinity ligand attached to a support matrix under conditions whereby the fragment antibody binds to the synthetic affinity ligand, wherein the synthetic affinity ligand has the formula: 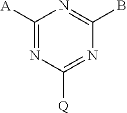
wherein
Q represents an attachment to a solid support matrix, optionally via a spacer group;
A and B are each independently —Y-phenyl or —Y-naphthyl groups substituted with one or more substituents capable of hydrogen bonding;
each Y independently represents —NR—, —O— or —S—; and
each R independently represents H or a C₁₋₄ alkyl group.

15. A process according to claim 14, wherein the substituents capable of hydrogen bonding are independently selected from —OH, —SH or —CO₂H groups.

16. A process according to claim 15, wherein the synthetic affinity ligand attached to a support matrix is selected from compounds of formula: 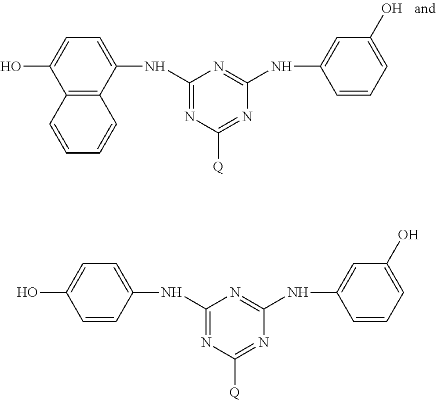
wherein Q represents an attachment to a solid support matrix, optionally via a spacer group.

17. A process according to claim 16, wherein Q represents a spacer group selected from the group consisting of groups of formula —NH—(CH₂)ₙNH-G;
—NH—(CH₂)ₙO-G; —O—(CH₂)ₙO-G; —O—(CH₂CH₂)ₙO-G; —NH—(CH₂)ₙO-G; and
—NH—(CH₂)ₙNH—(CH₂)ₓO-G
wherein n is a positive integer up to 12;
x is from 1 to 6; and
G is a solid support matrix.

18. A process according to claim 17, wherein n is from 2 to 6

19. A process according to claim 14, wherein the fragment antibody is bound to the ligand at a pH of from 6 to 8.

20. A process according to claim 19, wherein the fragment antibody is bound to the ligand at a pH of from 6.5 to 7.5.

21. A process according to claim 14, wherein the fragment antibody is bound to the ligand using a solution having an ionic strength of less than 50 mS/cm.

22. A process according to claim 21, wherein the fragment antibody is bound to the ligand using a solution having an ionic strength of between 10 and 40 mS/cm.

23. A process according to claim 14, wherein the fragment antibody is a Fab; an scFv; a single domain, or a fragment thereof; a bis scFv, Fab₂, Fab₃, minibody, diabody, triabody, tetrabody or tandab.

24. A process according to claim 23, wherein the fragment antibody is a single domain from a variable heavy chain or a fragment thereof, or a singe domain from a variable light chain or a fragment thereof.

25. A process for the preparation of a fragment antibody comprising:
(a) preparing a fragment antibody by recombinant technology to produce a medium comprising fragment antibody;
(b) separation of the fragment antibody from the medium by a process comprising contacting the medium comprising the fragment antibody with a synthetic affinity ligand attached to a support matrix under conditions whereby the fragment antibody binds to the synthetic affinity ligand, wherein the synthetic affinity ligand has the formula: 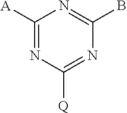
wherein
Q represents an attachment to a solid support matrix, optionally via a spacer group;
A and B are each independently —Y-phenyl or —Y-naphthyl groups substituted with one or more substituents capable of hydrogen bonding;
each Y independently represents —NR—, —O— or —S—; and
each R independently represents H or a C₁₋₄ alkyl group ; and
(c) releasing the fragment antibody from the affinity ligand.

26. A process according to claim 25, where the fragment antibody is produced by expression of an E. coli or Pichia pastoris host cell.

27. A process according to claim 25, wherein the substituents capable of hydrogen bonding are independently selected from —OH, —SH or —CO₂H groups.

28. A process according to claim 27, wherein the synthetic affinity ligand attached to a support matrix is selected from compounds of formula: 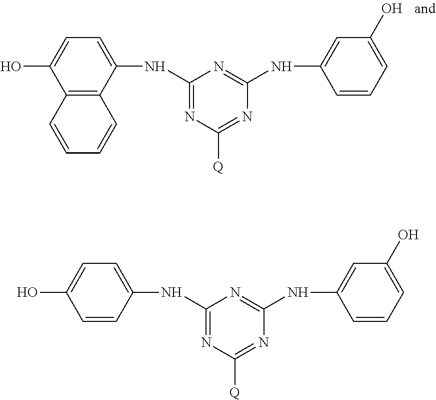
wherein Q represents an attachment to a solid support matrix, optionally via a spacer group.

29. A process according to claim 28 wherein Q represents a spacer group selected from the group consisting of groups of formula —NH—(CH₂)ₙNH-G;
—NH—(CH₂)ₙO-G; —O—(CH₂)ₙO-G; —O—(CH₂CH₂)ₙO-G; —NH—(CH₂)ₙO-G; and
—NH—(CH₂)ₙNH—(CH₂)ₓO-G
wherein n is a positive integer up to 12;
x is from 1 to 6; and
G is a solid support matrix.

30. A process according to claim 29, where n is from 2 to 6

31. A process according to claim 25, wherein the fragment antibody is bound to the ligand at a pH of from 6 to 8.

32. A process according to claim 31, wherein the fragment antibody is bound to the ligand at a pH of from 6.5 to 7.5.

33. A process according to claim 25, wherein the fragment antibody is bound to the ligand using a solution having an ionic strength of less than 50 mS/cm.

34. A process according to claim 33, wherein the fragment antibody is bound to the ligand using a solution having an ionic strength of between 10 and 40 mS/cm.

35. A process according to claim 25, wherein the fragment antibody is a Fab; an scFv; a single domain, or a fragment thereof; a bis scFv, Fab₂, Fab₃, minibody, diabody, triabody, tetrabody or tandab.

36. A process according to claim 35, wherein the fragment antibody is a single domain from a variable heavy chain or a fragment thereof, or a singe domain from a variable light chain or a fragment thereof.

37. A process for the preparation of a fragment antibody comprising:
(a) expression of the fragment antibody in an E. coli or Pichia pastoris host cell to produce a medium comprising fragment antibody;
(b) separation of the fragment antibody from the medium by a process comprising contacting the medium comprising the fragment antibody with a synthetic affinity ligand attached to a support matrix at a pH of from 6.5 to 7.5 in a solution having an ionic strength of between 10 and 40 mS/cm whereby the fragment antibody binds to the synthetic affinity ligand, wherein the synthetic affinity ligand has the formula: 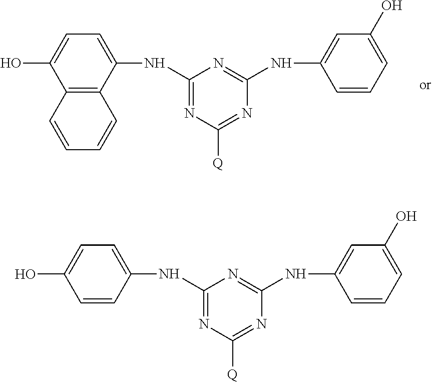
wherein Q represents a spacer group selected from the group consisting of groups of formula —NH—(CH₂)ₙNH-G;
—NH—(CH₂)ₙO-G; —O—(CH₂)ₙO-G; —O—(CH₂CH₂)ₙO-G; —NH—(CH₂)ₙO-G; and
—NH—(CH₂)ₙNH—(CH₂)ₓO-G
wherein n is from 2 to 6;
x is from 1 to 6; and
G is a solid support matrix; and
(c) releasing the fragment antibody from the affinity ligand.

38. A process according to claim 37, wherein the fragment antibody is a Fab; an scFv; a single domain, or a fragment thereof; a bis scFv, Fab₂, Fab₃, minibody, diabody, triabody, tetrabody or tandab.
